SEQUENCE LISTING (29 sequences)

SEQ ID NO: 1 - protein, 98 aa, Dechloromonas aromatica
  PEPTIDE (1)...(98): Dechloromonas aromatica strain RCB perchlorate reductase (PcrA), molybdopterin oxidoreductasemolybdopterin dinucleotide-binding region amino acids 105-202
GECAHHYMYGPHRLKYPLIRVGERGEGKWRRATWEEALDLISDKIIDTIKNHSPDCISVYSPLPGTAPVSFSAGHRFAHYIGAHTHTFFDWYSDHPTG

SEQ ID NO: 2 - protein, 98 aa, Dechloromonas agitata
  PEPTIDE (1)...(98): Dechloromonas agitata strain CKB perchlorate reductase alpha subunit, perchlorate reductase A catalytic subunit (PcrA) amino acids 105-202
GECAHDYMYGPHRLKYPLIRVGERGEGKWRRASWDEALDMIADKVVDTIKNHAPDCISVYSPVPAVAPVSFSAGHRFAHYIGAHTHTFFDWYGDHPTG

SEQ ID NO: 3 - protein, 96 aa, Ideonella dechloratans
  PEPTIDE (1)...(96): Ideonella dechloratans ATCC 51718 chlorate reductase molybdenum-containing subunit (ClrA) amino acids 114-209
GAVYCSWSKQPDHIKWPLKRVGERGERKWKRISWDEALTEIADKIIDTTVKRGPGNIYIPKRPFAVITNTAYTRMTKLLGAISPDATSMTGDLYTG

SEQ ID NO: 4 - protein, 96 aa, Thauera selenatis
  PEPTIDE (1)...(96): Thauera selenatis AX selenate reductase A molybdenum-containing subunit (SerA) amino acids 118-213
GAVYCSWSKQPDFLKYPLKRVGERGERKWKRISWDEAFTEIADKIIDTTVKRGPGNVCMPKRPFAVITSAGYSRLANLIGAIKPDVSSMTGDLYPG

SEQ ID NO: 5 - protein, 98 aa, Rhodovulum sulfidophilum
  PEPTIDE (1)...(98): Rhodovulum sulfidophilum strain SH1 dimethylsulfide dehydrogenase alpha subunit, dimethylsulfide dehydrogenase catalytic subunit containing molybdenum cofactor (DdhA) amino acids 112-209
GAIHSTSMYEADRLRYPMKRVGARGEGKWQRISWDQATEEIADKIIDIYEKYGPGKLMTHTGSGNMSMMRMAAPYRFASLVGGVQLDIFTDVGDLNTG

SEQ ID NO: 6 - protein, 98 aa, Azoarcus sp.
  PEPTIDE (1)...(98): Azoarcus sp. strain EB1 anaerobic ethylbenzene dehydrogenase subunit A (EbdA) amino acids 130-227
GSAFNNNLYGDERVKYPLKRVGKRGEGKWKRVSWDEAAGDIADSIIDSFEAQGSDGFILDAPHVHAGSIAWGAGFRMTYLMDGVSPDINVDIGDTYMG

SEQ ID NO: 7 - protein, 20 aa, Escherichia coli
  PEPTIDE (1)...(20): Escherichia coli K-12 nitrate reductase 1, alpha subunit (NarG) amino acids 96-115
GASYSWYLYSANRLKYPMMR

SEQ ID NO: 8 - protein, 78 aa, Escherichia coli
  PEPTIDE (1)...(78): Escherichia coli K-12 nitrate reductase 1, alpha subunit (NarG) amino acids 150-227
KQARGRGGFVRSSWQEVNELIAASNVYTIKNYGPDRVAGFSPIPAMSMVSYASGARYLSLIGGTCLSFYDWYCDLPPA

SEQ ID NO: 9 - protein, 92 aa, Unknown
  protein sequence of DNA extracted from Yolo silt loam soil enriched with 0.25 mM perchlorate and acetate (YA)
AHHYMYGPTRLKYPLIRVGERGEGKWRRASWDEALGMIADKIVDTIKTDGPDAISVYSPVPAVAPVSFSAGHRFAHLIGAHAHTFFDWYGDH

SEQ ID NO: 10 - protein, 92 aa, Unknown
  protein sequence from DNA extracted from Yolo silt loam soil enriched with 0.25 mM perchlorate and acetate (YA)
AHHYMYGPTRLKYPLIRVGERGEGKWRRASWDEALGMIADKIVDTIKTDGPDAISVYSPVPAVAPVSFAAGHRFAHLIGGHAHTYFDWYGDH

SEQ ID NO: 11 - protein, 92 aa, Dechloromonas aromatica
  PEPTIDE (1)...(92): Dechloromonas aromatica strain RCB perchlorate reductase (PcrA), molybdopterin oxidoreductasemolybdopterin dinucleotide-binding region amino acids 108-199
AHDYMYGPHRLKYPLIRVGERGEGKWRRASWDEALDMIADKVVDTIKNHAPDCISVYSPVPAVAPVSFSAGHRFAHYIGAHTHTFFDWYGDH

SEQ ID NO: 12 - protein, 92 aa, Dechloromonas sp.
  PEPTIDE (1)...(92): Dechloromonas sp. strain MissR perchlorate reductase alpha subunit (PcrA) partial CDS translation
AHHYMYGPHRIKYPLIRVGERGEGKWRRASWEEALDMIADKCVDTIKNHAPDCISVFSPVPAVSPVSFSAGHRFAHYIGAHTHTFFDWYGDH

SEQ ID NO: 13 - protein, 92 aa, Dechloromonas agitata
  PEPTIDE (1)...(92): Dechloromonas agitata strain CKB perchlorate reductase alpha subunit, perchlorate reductase A catalytic subunit (PcrA) amino acids 108-199
AHHYMYGPHRLKYPLIRVGERGEGKWRRATWEEALDLISDKIIDTIKNHSPDCISVYSPLPGTAPVSFSAGHRFAHYIGAHTHTFFDWYSDH

SEQ ID NO: 14 - protein, 92 aa, Azospirillum sp.
  PEPTIDE (1)...(92): Azospirillum sp. strain TTI perchlorate reductase alpha subunit (PcrA) partial CDS translation
AHHYMYGPHRLKYPLIRVGERGEGKWRRATWDEALELIAEKVIDTIRNHAPDCISVYSPGPSVAPVSFAAGHRFAHLIGGHAHTYFDWYGDH

SEQ ID NO: 15 - protein, 92 aa, Unknown; identical to SEQ ID NO: 14
  protein sequence from DNA extracted from Yolo silt loam soil enriched with 0.25 mM perchlorate and acetate (YA) and protein sequence from DNA extracted from Yolo silt loam soil enriched with 0.25 mM perchlorate and hydrogen (YH)

SEQ ID NO: 16 - protein, 92 aa, Unknown
  protein sequence from DNA extracted from Yolo silt loam soil enriched with 0.25 mM perchlorate and hydrogen (YH)
AHHYMYGPHRLKYPLIRVGERGEGKWRRATWDEALGLIANKIVDTIQNHAPDCISVYSPVPAVAPVSFAAGHRFAHLIGGHTHTFFDWYGDH

SEQ ID NO: 17 - protein, 92 aa, Dechlorospirillum sp.
  PEPTIDE (1)...(92): Dechlorospirillum sp. strain WD perchlorate reductase alpha subunit (PcrA) partial CDS translation
AHHYMYGPYRIKHPLIRIGQRGEGKWRRASWEEALQLIADRTVDALVRHGPDTISVFSPSPAVAPVSYAAGHRFAHLIGAHAHTFFDWYGDH

SEQ ID NO: 18 - protein, 92 aa, Unknown
  protein sequence from DNA extracted from Yolo silt loam soil enriched with 0.25 mM perchlorate and acetate (YA)
AHHYMYGPYRVKHPLIRVGERGEGRWRRATWDEALQLIADRALDTMLHHGPEAIGVFSPAPAVAPVSYAAGHRFAHLIGAHTHTFYDWYGDH

SEQ ID NO: 19 - protein, 92 aa, Escherichia coli
  PEPTIDE (1)...(92): Escherichia coli K-12 nitrate reductase 1, alpha subunit (NarG) amino acids 99-224
YSWYLYSANRLKYPMMRKQARGRGGFVRSSWQEVNELIAASNVYTIKNYGPDRVAGFSPIPAMSMVSYASGARYLSLIGGTCLSFYDWYCDL

SEQ ID NO: 20 - protein, 92 aa, Haloarcula marismortui
  PEPTIDE (1)...(92): Haloarcula marismortui nitrate reductase subunit 1, nitrate reductase alpha subunit (NarG)
YTDYVNADQRVLHPLRRTGERGEGQWERISWDEALTEIADHVIDEVQAGRYDAISGFTPIPAMSPVSFASGSRLVNLLGGVSHSFYDWYSDL

SEQ ID NO: 21 - protein, 90 aa, Ideonella dechloratans
  PEPTIDE (1)...(90): Ideonella dechloratans ATCC 51718 chlorate reductase molybdenum-containing subunit, chlorate reductase subunit alpha (ClrA) amino acids 117-206
YCSWSKQPDHIKWPLKRVGERGERKWKRISWDEALTEIADKIIDTTVKRGPGNIYIPKRPFAVITNTAYTRMTKLLGAISPDATSMTGDL

SEQ ID NO: 22 - protein, 90 aa, Thauera selenatis
  PEPTIDE (1)...(90): Thauera selenatis AX selenate reductase A molybdenum-containing subunit, reductase A subunit alpha (SerA) amino acids 121-210
YCSWSKQPDFLKYPLKRVGERGERKWKRISWDEAFTEIADKIIDTTVKRGPGNVCMPKRPFAVITSAGYSRLANLIGAIKPDVSSMTGDL

SEQ ID NO: 23 - protein, 92 aa, Rhodovulum sulfidophilum
  PEPTIDE (1)...(92): Rhodovulum sulfidophilum strain SH1 dimethylsulfide dehydrogenase alpha subunit, dimethylsulfide dehydrogenase catalytic subunit containing molybdenum cofactor (DdhA, DMS DH) amino acids 115-206
HSTSMYEADRLRYPMKRVGARGEGKWQRISWDQATEEIADKIIDIYEKYGPGKLMTHTGSGNMSMMRMAAPYRFASLVGGVQLDIFTDVGDL

SEQ ID NO: 24 - protein, 92 aa, Azoarcus sp.
  PEPTIDE (1)...(92): Azoarcus sp. strain EB1 anaerobic ethylbenzene dehydrogenase subunit A (EbdA) amino acids 133-224
FNNNLYGDERVKYPLKRVGKRGEGKWKRVSWDEAAGDIADSIIDSFEAQGSDGFILDAPHVHAGSIAWGAGFRMTYLMDGVSPDINVDIGDT

SEQ ID NO: 25 - protein, 92 aa, Escherichia coli
  PEPTIDE (1)...(92): Escherichia coli K-12 nitrate reductase, periplasmic, large subunit, nitrate reductase 3 (NapA)
LPKIMYGKDRLTQPLLRGKYDKEGEFTPITWDQAFDVMEEKFKTALKEKGPESIGMFGSGQWTIWEGYAASKLFKAGFRSNNIDPNARHCMA

SEQ ID NO: 26 - protein, 6 aa, Artificial Sequence
  exemplary amino acid sequence encoded by degenerate oligonucleotide
YEILLK

SEQ ID NO: 27 - DNA, 18 bp, Artificial Sequence
  degenerate oligonucleotide encoding exemplary amino acid sequence
taygarathctnctnaar

SEQ ID NO: 28 - DNA, 25 bp, Artificial Sequence
  synthetic primer pcrA320F to identify PcrA sequence regions
gcgcccaccactacatgtayggncc

SEQ ID NO: 29 - DNA, 22 bp, Artificial Sequence
  synthetic primer pcrA598R to identify PcrA sequence regions
ggtggtcgccgtaccartcraa

(19) United States
(12) Patent Application Publication
(10) Pub. No.: US 2009/0258349 A1
(43) Pub. Date: Oct. 15, 2009

(54) METHOD FOR DETECTING AND QUANTIFYING PERCHLORATE-REDUCING BACTERIA

(75) Inventors: Krassimira R. Hristova, Woodland, CA (US); Mamie Nozawa-Inoue, Irvine, CA (US); Katie M. Scow, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 11/868,895

(22) Filed: Oct. 8, 2007

Related U.S. Application Data
(60) Provisional application No. 60/850,039, filed on Oct. 6, 2006.

(51) Int. Cl. C12Q 1/68

(52) U.S. Cl. 435/6

(57) ABSTRACT

The present invention provides methods, compositions, and kits for detecting and quantitating perchlorate-reducing bacteria in samples using reagents that hybridize to and allow amplification of the pcrA gene. The invention includes a quantitative real-time PCR assay for amplification of the pcrA gene which may be used to detect and quantitate perchlorate-reducing bacteria in samples.

### CROSS-REFERENCES TO RELATED APPLICATIONS

The present application claims priority to U.S. Ser. No. 60/850,039, filed Oct. 6, 2006, herein incorporated by reference in its entirety.

### STATEMENT AS TO RIGHTS TO INVENTIONS MADE UNDER FEDERALLY SPONSORED RESEARCH OR DEVELOPMENT

This invention was made with Government support under Grant No. 5 P42 ES04699 from the National Institute of Environmental Health Sciences (NIEHS) of the NIH. The Government has certain rights in this invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Perchlorate (ClO₄⁻) is a widespread environmental contaminant that disrupts thyroid gland function (Motzer, W. E., Environ. Forensics, 2:301-3 11 (2001); Urbansky, E. T., Environ. Sci. Pollut. Res., 9:187-192 (2002)). According to a recent U.S. EPA report, contamination of groundwater, surface water, and soil by perchlorate has been detected in 35 states, with California reporting the largest number of detections (U.S. EPA, Known perchlorate releases in the U.S.—Mar. 25, 2005 (2005)). Bacterial strains capable of respiratory perchlorate reduction, a process that produces innocuous chloride, have been isolated from a variety of sources (Bruce, R. A. et al., Environ. Microbiol., 1:319-329 (1999); Coates, J. D. et al., Appl. Environ. Microbiol., 65:5234-5241 (1999); Herman, D. C., and W. T. Frankenberger, J. Environ. Qual., 28:1018-1024 (1999); Logan, B. E. et al., Appl. Environ. Microbiol., 67:2499-2506 (2001); Rikken, G. B. et al., Appl. Microbiol. Biotechnol., 45:420-426 (1996); Shrout, J. D. et al., Appl. Microbiol. Biotechnol., 67:261-268 (2005); Wallace, W. et al., J. Ind. Microbiol., 16:68-72 (1996); Waller, A. S. et al., Environ. Microbiol., 6:517-527 (2004); Zhang, H. S. et al., Environ. Microbiol., 4:570-576 (2002)). Although these perchlorate-reducing bacteria (PRB) appear to be ubiquitous (Coates, J. D. et al., Appl. Environ. Microbiol., 65:5234-5241 (1999)), our knowledge of their population dynamics in the environment is very limited.

Estimating the abundance and growth of PRB can be helpful in assessing the potential for and optimization of biological treatment, a promising technology for perchlorate remediation. Quantitative information, however, is limited mostly to pure culture studies (Logan, B. E. et al., Appl. Environ. Microbiol., 67:2499-2506 (2001); Rikken, G. B. et al., Appl. Microbiol. Biotechnol., 45:420-426 (1996); Waller, A. S. et al., Environ. Microbiol., 6:517-527 (2004)), with few techniques available for enumerating PRB within larger microbial communities. Existing culture-dependent MPN methods require a several month incubation time to develop estimates of numbers of PRB in soil and water samples (Coates, J. D. et al., Appl. Environ. Microbiol., 65:5234-5241 (1999); Wu, J. et al., Bioremed. J., 5:119-130 (2001)).

More rapid detection could be achieved by targeting functional genes common to this bacterial group, such as genes encoding perchlorate reductase (pcrABCD) (Bender, K. S. et al., J. Bacteriol., 187:5090-5096 (2005)) and chlorite dismutase (cld) (Bender, K. S. et al., Appl. Environ. Microbiol., 68:4820-4826 (2002); Bender, K. S. et al., Appl. Environ. Microbiol., 70:5651-5658 (2004)). These two enzymes catalyze reactions of perchlorate to chlorite (ClO₂⁻) (Kengen, S. W. M. et al., J. Bacteriol., 181:6706-6711 (1999)) and chlorite to chloride (van Ginkel, C. G. et al., Arch. Microbiol., 166:321-326 (1996)), respectively. A nested PCR assay targeting the cld gene has been developed and applied to environmental samples (Bender, K. S. et al., Appl. Environ. Microbiol., 70:5651-5658 (2004)). However, the cld gene is not specific to PRB, because non-PRB such as chlorate (ClO₃⁻)-reducing bacteria also possess cld genes (Stenklo, K. et al., J. Biol. Inorg. Chem., 6:601-607 (2001); Wolterink, A. et al., Int. J. Syst. Evol. Microbiol., 52:2183-2190 (2002)). Targeting the perchlorate reductase is more appropriate for detecting PRB, because the pcr gene appears to be exclusively present in PRB, and the enzyme catalyzes the rate-limiting step in perchlorate reduction (Rikken, G. B. et al., Appl. Microbiol. Biotechnol., 45:420-426 (1996)). A slot-blot hybridization probe has been designed for the pcrA gene, encoding the catalytic subunit of perchlorate reductase (Bender, K. S. et al., J. Bacteriol., 187:5090-5096 (2005)).

Thus, specific and sensitive assays are needed to detect and quantitate PRB, particularly in environmental samples. The present invention satisfies these and other needs.

### BRIEF SUMMARY OF THE INVENTION

The present invention provides a specific and sensitive assay for detecting PRB in a variety of samples using methods that rely on PCR amplification of the pcrA gene. In one embodiment, the invention utilizes a real-time quantitative PCR (qPCR) assay, based on amplification of the pcrA gene, for quantitatively detecting PRB in environmental samples. The invention is based in part on the identification of primers that may be used to hybridize to and specifically amplify the pcrA gene from PRB, while avoiding hybridization to and amplification of sequences corresponding to other enzymes of related sequence, but with distinct functions, such as other bacterial reductases, for example the DMSO reductases.

Accordingly, in one embodiment, the present invention provides a method of detecting perchlorate-reducing bacteria in a sample by contacting the sample with an oligonucleotide that specifically hybridizes to a subsequence of the pcrA gene, whereby specific hybridization to the subsequence indicates the presence of perchlorate-reducing bacteria in the sample. In an aspect of this embodiment, the oligonucleotide is pcrA320F or pcrA598R. In another aspect, oligonucleotides comprising pcrA320F and pcrA598R are used. Furthermore, the oligonucleotides can include a detectable label, which can be a fluorescent label. In a further aspect, the method of detecting is carried out using PCR, which in some instances, can be real-time quantitative PCR. In some aspects, the method is carried out using a soil sample.

In another embodiment, the present invention provides a composition for detecting perchlorate-reducing bacteria in a sample, where the composition includes an oligonucleotide that specifically hybridizes to a subsequence of the pcrA gene. In an aspect of this embodiment, the oligonucleotide is pcrA320F or pcrA598R. In another aspect, oligonucleotides comprising pcrA320F and pcrA598R are used. Furthermore, the oligonucleotides can include a detectable label, which can be a fluorescent label.

In a yet another embodiment, the present invention provides a kit for detecting perchlorate-reducing bacteria in a sample, where the kit comprises an oligonucleotide that specifically hybridizes to a subsequence of the pcrA gene. In an aspect of this embodiment, the oligonucleotide is pcrA320F or pcrA598R. In another aspect, oligonucleotides comprising pcrA320F and pcrA598R are used. Furthermore, the oligonucleotides can include a detectable label, which can be a fluorescent label. A further component of the kit can be a DNA polymerase, which can be Taq polymerase.

### BRIEF DESCRIPTION OF THE DRAWINGS

FIG. 1. Multiple sequence alignment for perchlorate reductase and related molybdoenzymes. The region encompassed by the primers used for the qPCR assay is shown, and the residues within the PcrA protein sequences corresponding to the primer DNA sequences are boxed. Positions with identical residues in all seven sequences are shaded black, and positions with identical residues in all sequences except NarG are shaded gray. The conserved aspartyl residue that provides a side-chain ligand to Mo in the molybdenum cofactor corresponds to NarG residue Asp-223 (Jormakka, M. et al., Structure, 12:95-104 (2004)). The NarG sequence contains a 34-residue region that is not present in the other sequences (indicated by three dots). Sequences are PcrA, perchlorate reductase (sequence 1: Dechloromonas agitata CKB; GenBank accession number AY180108; sequence 2: D. aromatica RCB; AAZ47315), ClrA, chlorate reductase (Ideonella dechloritans ATCC 51718; CAD97447), Selenate reductase (Thauera selenatis AX; AJ007744), DdhA, dimethylsulfide dehydrogenase (Rhodovulum sulfidophilum SH1; AF453-479), EbdA, ethylbenzene dehydrogenase (Azoarcus sp. EB1; AF337952), and NarG, nitrate reductase (Escherichia coli K-12, NP 415742).

FIG. 2. Multiple sequence alignment for additional perchlorate reductase genes and related molybdoenzymes. YA1-4 and YH1-2 refer to sequences recovered from Yolo silt loam enriched with perchlorate and acetate (YA) or hydrogen (YH). The numbers of clones with identical sequences, in total 19 and 20 clones from the soil enrichments YA and YH, respectively, are shown in parentheses. Sequences are Dar_RCB_PcrA: D. aromatica RCB PcrA; Dechl_MissR_PcrA: Dechloromonas sp. MissR PcrA; Dag_CKB_PcrA: Dechloromonas agitata CKB PcrA; Azsprllm_TTI_PcrA: Azospirillum sp. TTI PcrA; Dechlrsprllm_WD_PcrA: Dechlorospirillum sp. WD PcrA; Eco_NarG: Escherichia coli K-12, NarG; Ide_ClrA: Ideonella dechloritans ATCC 51718 ClrA; Tse_SerA: Thauera selenatis SerA; Rsu_DdhA: Rhodovulum sulfidophilum SH1 DdhA; Azo_DbdA: Azoarcus sp. EB1 DbdA; Eco_NapA: NapA (nitrate reductase).

FIG. 3. Phylogenetic tree of deduced PcrA protein sequences and reference sequences. Bootstrap values above 50 from 100 resampling are shown at each node. The sequences obtained in this study are indicated in bold type. The numbers of clones with identical sequences, in total 19 and 20 clones from the soil enrichments YA and YH, respectively, are shown in parentheses. The GenBank accession numbers for reference sequences are shown in FIG. 1 caption, except Escherichia coli NapA (AAC75266). The scale bar indicates 0.2 changes per amino acid.

FIG. 4. Standard curve relating pcrA gene copy numbers and qPCR threshold cycles (CT) using a plasmid containing D. agitata pcrA gene (mean±SD, n=6).

### DETAILED DESCRIPTION OF THE INVENTION

### I. Introduction

The present invention is based in part on the identification of oligonucleotide primers that may be used to hybridize to and specifically amplify the pcrA gene from PRB, while avoiding hybridization to and amplification of sequences corresponding to other enzymes of related sequence, but with distinct functions, such as other bacterial reductases, for example the DMSO reductases. Oligonucleotide primers were designed by identifying regions of amino acid sequence which showed strong conservation among known pcrA genes, but which were divergent in the sequences of other reductase genes. Accordingly, the oligonucleotide primers of the invention may be used in specific and sensitive hybridization and amplication based assays for the detection of PRB in samples. In one embodiment, the invention utilizes a real-time quantitative PCR (qPCR) assay, based on amplification of the pcrA gene, for quantitatively detecting PRB in environmental samples, such as perchlorate contaminated soil samples.

Thus, the invention provides compositions, methods, and kits for the detection and quantitation of PRB that may be applied to a variety of samples of interest. Among other uses, when applied to samples such as perchlorate contaminated soil, the present invention may be used to determine the abundance and growth of PRB to assess and optimize biological approaches to remediation utilizing PRB.

### II. Definitions

The terms “prechlorate-reducing bacteria” or “PRB” are used interchangeably and refer generally to any bacteria having the pcrA gene. Examples of such bacteria include Dechloromonas agitate and Dechloromonas aromatica, among others.

The pcrA gene encodes a subunit of the protein, perchlorate reductase, which catalyzes the reduction of perchlorate (ClO₄⁻) to chlorite (ClO₂⁻). The pcrA gene is a member of the perchlorate reductase operon that comprises the genes pcrABCD, which has been identified in perchlorate-reducing bacteria, including D. agitate and D. aromatica. The α-subunit of perchlorate reductase is encoded by the pcrA gene. The PcrA protein is an electron transfer protein of approximately 100 kDa possessing a pterin molybdenum cofactor and iron-sulfur centers. Analysis of the amino acid sequence of PcrA reveals that it is a 927 amino acid protein containing a molybdopterin-binding domain and a twin-arginine signal motif. The reaction catalyzed by perchlorate reductase is the reduction of ClO₄⁻/ClO₃⁻ to ClO₂⁻. The subsequent action of chlorite dismutase (Cld) converts ClO₂⁻ into harmless Cl⁻.

A number of nucleotide and protein sequences corresponding to the pcrA gene and its encoded protein, PcrA, are known, including those from Dechloromonas agitate CKB and Dechloromonas aromatica, which have the accession numbers: AY180108, AAO49008, and AAZ47315.

An “amplification reaction” refers to any chemical reaction, including an enzymatic reaction, which results in increased copies of a template nucleic acid sequence. Amplification reactions include polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20(7):1691 (1992); Walker PCR Methods Appl 3(1):1 (1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), rolling circle amplification (RCA) (Lisby, Mol. Biotechnol. 12(1):75 (1999)); Hatch et al., Genet. Anal. 15(2):35 (1999)) and branched DNA signal amplification (bDNA) (see, e.g., Iqbal et al., Mol. Cell Probes 13(4):315 (1999)).

“Amplifying” refers to submitting a solution to conditions sufficient to allow for amplification of a polynucleotide. Components of an amplification reaction include, e.g., primers, a polynucleotide template, polymerase, nucleotides, and the like.

“Amplification reagents” refer to reagents used in an amplification reaction. These reagents can include, e.g., oligonucleotide primers; borate, phosphate, carbonate, barbital, Tris, etc. based buffers (see, U.S. Pat. No. 5,508,178); salts such as potassium or sodium chloride; magnesium; deoxynucleotide triphosphates (dNTPs); a nucleic acid polymerase such as Taq DNA polymerase; as well as DMSO; and stabilizing agents such as gelatin, bovine serum albumin, and non-ionic detergents (e.g. Tween-20).

The term “primer” refers to a nucleic acid sequence that primes the synthesis of a polynucleotide in an amplification reaction. Typically a primer comprises fewer than about 100 nucleotides and preferably comprises fewer than about 30 nucleotides. Exemplary primers range from about 5 to about 25 nucleotides.

A “degenerate” primer refers generally to an oligonucleotide primer comprising a mixture of similar, but not identical sequences. Frequently, degenerate primers are used to encode the multiplicity of codons that arise from the degeneracy of the genetic code or to allow a codon position in a primer to encode multiple amino acids that share a similar triplet code of bases. Thus, for instance to encode the amino acid sequence, YEILLK, the degenerate oligonucleotide: TA(T or C)GA(A or G)AT(C or T or A)CT(C or T or A or G)CT(C or T or A or G)AA(A or G) might be designed. The term degenerate primer also includes primers in which inosine “I” is included to allow a particular base position in the primer to hydrogen bond to any one of the 4 bases that may be present in the target DNA to which the primer hybridizes. The skilled artisan will appreciate that all or only particular positions within a primer sequence need be degenerate depending on the application of the primer.

As used herein a “nucleic acid probe” or “oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “subsequence” refers to a sequence of nucleotides that are contiguous within a second sequence but does not include all of the nucleotides of the second sequence.

A “target” or “target sequence” refers to a single or double stranded polynucleotide sequence sought to be amplified in an amplification reaction.

The phrase “nucleic acid” or “polynucleotide” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Two nucleic acid sequences or polypeptides are said to be “identical” if the sequence of nucleotides or amino acid residues, respectively, in the two sequences is the same when aligned for maximum correspondence as described below. The term “complementary to” is used herein to mean all of a first sequence is complementary to at least a portion of a reference polynucleotide sequence.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The phrase “selectively (or specifically) hybridizes to” refers to the binding, duplexing, or hybridizing of a molecule only to a particular nucleotide sequence under stringent hybridization conditions when that sequence is present in a complex mixture.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. A positive hybridization is at least twice background. Those of ordinary skill will readily recognize that alternative hybridization and wash conditions can be utilized to provide conditions of similar stringency. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman PNAS USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. The percent identity between two sequences can be represented by any integer from 25% to 100%. More preferred embodiments include at least: 25%, 30%, 35%, 40%, 45%, 50%, 55%, 60%, 65%, 70%, 75%, 80%, 85%, 90%, 95%, or 99%.

One example of an algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, PNAS USA 89:10915 (1989)) alignments (B) of 50, expectation (E) of 10, M=5, N=−4, and a comparison of both strands.

### III. Overview of Detection of Perchlorate-Reducing Bacteria

In order to specifically detect the presence of PRB in a sample, a functional gene common to this group of bacteria was selected for the development of a PCR based amplification method of detection. For this purpose, the pcrA gene, which is the first gene in the perchlorate reductase (pcr) operon was chosen. The protein encoded by pcrA encodes one of the two structural subunits of perchlorate reductase. Previous attempts to develop a PCR based assay to specifically detect PRB, such as by using nested PCR targeting of the chlorite dismutase gene (cld), were unsuccessful because this gene was also present in non-PRB such as chlorate-reducing bacteria. The perchlorate reductase gene is a more appropriate candidate for a PCR based assay as this enzyme appears to be exclusively present in PRB and catalyzes the rate-limiting step in perchlorate reduction.

Because amino acid sequence analysis of perchlorate reductase subunits indicated that this protein had high sequence similarity to enzymes of the DMSO reductase family, such as nitrate reductase, selenate reductase, dimethyl sulfide dehydrogenase, ethylbenzene dehydrogenase, and chlorate reductase, primers for amplification were designed which specifically amplified the pcrA gene while avoiding amplification of other related, but distinct, reductase genes. As described below in Example 1, primer selection was performed by aligning the amino acid sequences of known PcrA proteins with those of other related, but distinct, reductases. Regions of high sequence homology among the PcrA sequences but which had low sequence homology to other related, but distinct, reductases were identified. Particular attention was paid to regions of the alignment that corresponded to functional regions of the PcrA protein, as these would likely be unique to this class of enzyme and be conserved in PcrA proteins across different genera of bacteria with perchlorate reduction activity. The identified amino acid sequences were reverse translated into their corresponding nucleotide sequences. Degenerate oligonucleotide primers were designed to take into account amino acid differences across the PcrA protein sequences and alternative codon usages in different PRB. The final primer sets were chosen taking into consideration factors such as the spacing between the primers, the level of degeneracy of particular primer sets, and the Tₘ of the primers, among other considerations.

The primer sets chosen can then be used in any of a variety of PCR based amplification and detection methods as described generally below and with greater specificity in the Examples.

### IV. Methods of the Invention

A. Nucleic Acid Extraction

Nucleic acids can be extracted from samples using any method known in the art and/or commercially available kits. In the case of soil samples, a number of procedures for the isolation of DNA from soil samples are known in the art. See, e.g., Kauffmann, I. M. et al., Appl Microbiol Biotechnol., 64: 665-70 (2004); Bertrand H. et al., J. Microbiol. Methods, 62: 1-11 (2005); Porteous L. A. et al., Curr. Microbiol., 27: 115-118 (1993). Additionally, commercially available kits for DNA isolation from soil are also available, such as the UltraClean Microbial DNA kit (MoBio Laboratories, Carlsbad, Calif.) and FastDNA Spin kit for soil (MP Biomedicals, Solon, Ohio). Nucleic acids from bacterial cultures can be isolated using conventional methods developed for DNA isolation from bacteria, such as those described in Sambrook, et al., infra.

Basic texts disclosing the general methods of use in this invention include MOLECULAR CLONING: A LABORATORY MANUAL (Sambrook et al. eds. 3d ed. 2001); PCR PROTOCOLS: A GUIDE TO METHODS AND Applications (Innis et al., eds, 1990); GENE TRANSFER AND EXPRESSION: A LABORATORY MANUAL (Kriegler, 1990); and CURRENT PROTOCOLS IN MOLECULAR BIOLOGY (Ausubel et al., eds., 1994)).

B. Primer Design

Basic considerations that are employed in the design of primers for hybridization and PCR generally take into account factors such as: primer length, base pairing of the terminal nucleotide of a PCR primer with its target sequence, G/C content and Tₘ, and PCR product length and location within a target sequence.

The specificity of a PCR reaction is generally controlled by the length of the primer and the annealing temperature of the PCR reaction. It has been found that oligonucleotides between 18 and 24 bases tend to be very sequence specific if the annealing temperature of the PCR reaction is set within a few degrees of the primer Tₘ. However, longer primers (e.g., 25-35 bases) may be desirable in cases in which heterogeneity is expected. Such situations include applications in which one desires to amplify sequences encoding closely related molecules, such as isoforms of a protein or proteins within a family, homoglous genes from different species, or when a significant degree of sequence heterogeneity is expected (i.e., viral genomes which undergo rapid mutation). For such applications, one frequently compares all available related sequences and determines the DNA or protein region of least sequence variability. These regions serve as the starting point for primer selection. In many instances, the researcher will know the function of a protein of interest and the domains of the protein necessary for that function. In such cases, a comparison of the available sequences in the critical regions for function can be used to aid in the design of primers for PCR. In this way, primers can be designed around conserved sequences.

The placement of the 3′ end of the primer is critical for a successful PCR reaction because the 3′-terminal position in the primer is essential for controlling mispriming. Thus, well conserved amino acids within an alignment are preferred as a basis for designing the 3′ end of a primer.

In general, PCR primers should have a G/C content that provides a Tₘ that allows for efficient annealing at the temperatures typically used for PCR. For example, oligonucleotides of 20 bases in length with a 50% G/C content generally have Tₘ values in the range of 56-62° C. Generally, the Tm of the individual primers in a primer pair should be well matched.

Additionally, selection of primers will take into account the relative position of hybridization of the primers within a template as this determines the length of the resulting PCR product. The length of a PCR product that can be efficiently amplified will be dependent in part on the nature of the template material. Heterogenous material, such as fixed clinical material, tends not to support the amplification of longer PCR products. In contrast, purified plasmid DNA or high molecular DNA can be used to routinely obtain products of greater than 3 kb. For the purposes of detection, PCR products of 150-1000 bp are frequently desirable. For the purposes of the development of a routine clinical or field assay, a smaller amplification product of 100-300 bp may be desirable.

For some applications, such as when amplification of a related, but not identical sequence is desired, degenerate oligonucleotide primers may be used. Degenerate primers comprise a mixture of similar but not identical primers. Frequently, degenerate oligonucleotides are used if a particular gene is to be amplified from different organisms, because the particular gene in different organisms is likely to be similar but not identical. Another use for degenerate primers is when primer design is based on protein sequence since several different codons can code for one amino acid; thus, it is often difficult to deduce which codon is used in a particular case. As an example, owing to the degeneracy of the genetic code, proline can be encoded by any one of four related codons: CCC, CCG, CCA, and CCT.

The degeneracy of a primer set is calculated by multiplying together the number of possibilities at each position of a primer at which multiple bases are present. Thus, the degeneracy of the sequence: TA(T or C)GA(A or G)AT(C or T or A)CT(C or T or A or G)CT(C or T or A or G)AA(A or G) is 2×2×3×4×4×2=384-fold degeneracy. Thus, this primer will be a mixture of 384 primers. In order to reduce the level of degeneracy, codon usage tables for different organisms may be used to select for favored codons corresponding to a particular amino acid. Alternatively, an inosine residue, which is capable of hydrogen bonding to any of the four bases, may be inserted into positions of a primer which have multiple possible bases. Preferably the degree of degeneracy should be less than 500, less than 400, less than 300, less than 200, less than 100, less than 50, or less than 25.

A variety of computer programs are available to aid in the design of PCR primers, many of which are available online (see, e.g., Abd-Elsalam, African J. Biotech., 2: 91-95 (2003)). Among these, the program CODEHOP (COnsensus DEgenerate Hybrid Oligonucleotide Primers) is useful for designing degenerate oligonucleotide primers based on sequence alignments (see Rose, T. M. et al., Nucleic Acids Res., 26: 1628-1635 (1998)).

The primers derived from the use of CODEHOP comprise a short 3′ degenerate core region and a longer 5′ consensus clamp region. Only 3-4 highly conserved amino acid residues are necessary for design of the core, which is stabilized by the clamp during annealing to template molecules. During later rounds of amplification, the non-degenerate clamp permits stable annealing to product molecules.

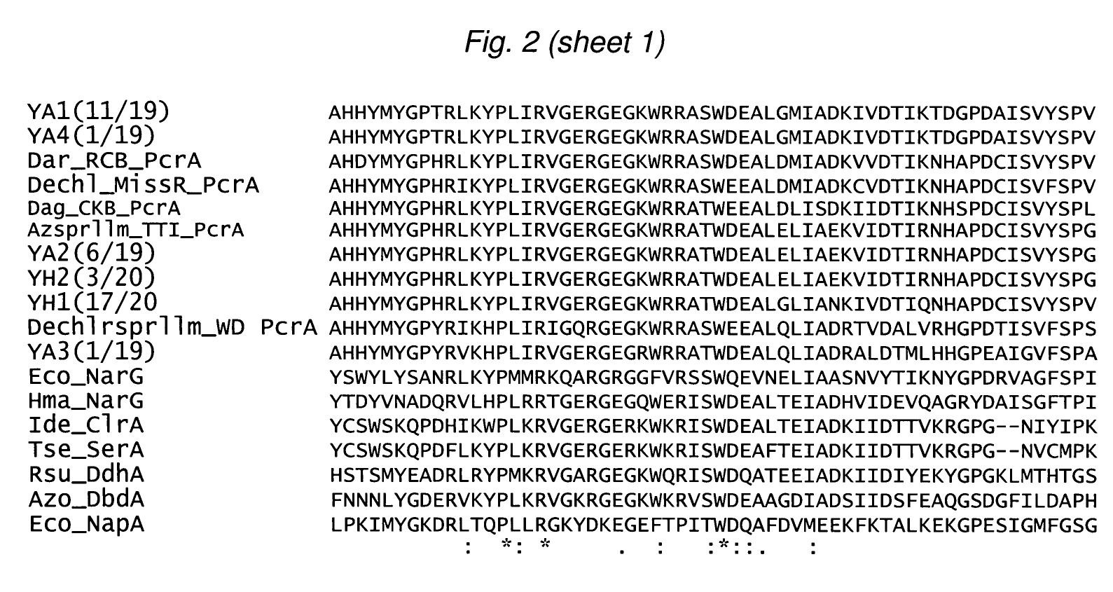

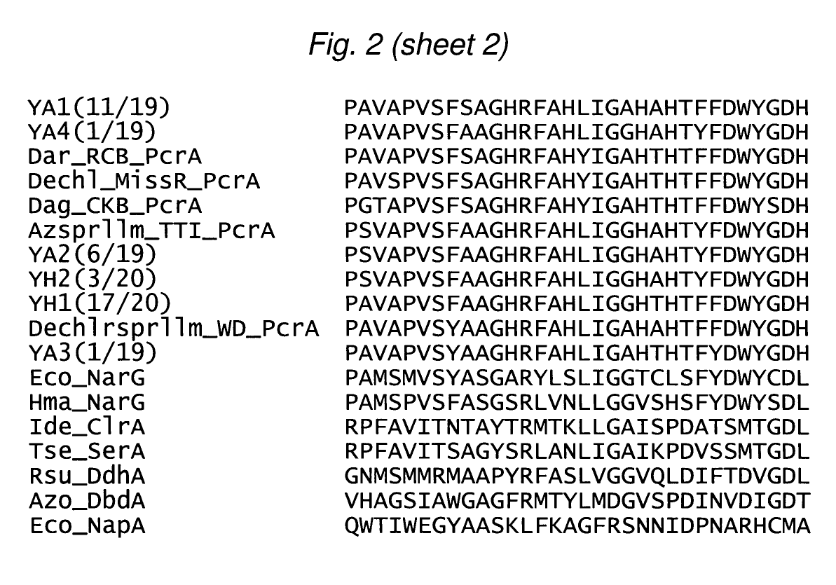

The skilled artisan will recognize that in the context of the present invention, as additional sequence information corresponding to members of a particular protein family are generated using an initial set of primers (such as those shown in FIG. 2), the newly derived sequence information can be used to design further primers.

C. Amplification Reaction Components

1. Oligonucleotides

Generally, the oligonucleotides that are used in the present invention can be chemically synthesized, using methods known in the art. These oligonucleotides can be labeled with radioisotopes, chemiluminescent moieties, or fluorescent moieties. Such labels are useful for the characterization and detection of amplification products using the methods and compositions of the present invention.

Typically, the target primers are present in the amplification reaction mixture at a concentration of about 0.1 μM to about 1.0 μM, more typically about 0.25 μM to about 0.9 μM, even more typically about 0.5 to about 0.75 μM, most typically about 0.6 μM. The primer length can be about 8 to about 100 nucleotides in length, more typically about 10 to about 75 nucleotides in length, more typically about 12 to about 50 nucleotides in length, more typically about 15 to about 30 nucleotides in length, most typically about 19 nucleotides in length. Preferably, the primers of the invention all have approximately the same melting temperature. Typically, the primers amplify a sequence of the pcrA gene while not amplifying the sequence of sequence-similar, but functionally distinct genes.

2. Buffer

Buffers that may be employed are borate, phosphate, carbonate, barbital, Tris, etc. based buffers. (See, U.S. Pat. No. 5,508,178). The pH of the reaction should be maintained in the range of about 4.5 to about 9.5. (See, U.S. Pat. No. 5,508,178. The standard buffer used in amplification reactions is a Tris based buffer between 10 and 50 mM with a pH of around 8.3 to 8.8. (See Innis et al., supra.).

One of skill in the art will recognize that buffer conditions should be designed to allow for the function of all reactions of interest. Thus, buffer conditions can be designed to support the amplification reaction as well as any subsequent manipulations, such as e.g., restriction enzyme reactions. A particular reaction buffer can be tested for its ability to support various reactions by testing the reactions both individually and in combination.

3. Salt Concentration

The concentration of salt present in the reaction can affect the ability of primers to anneal to the target nucleic acid. (See, Innis et al.). Potassium chloride can added up to a concentration of about 50 mM to the reaction mixture to promote primer annealing. Sodium chloride can also be added to promote primer annealing. (See, Innis et al.).

4. Magnesium Ion Concentration

The concentration of magnesium ion in the reaction can affect amplification of the target sequence(s). (See, Innis et al.). Primer annealing, strand denaturation, amplification specificity, primer-dimer formation, and enzyme activity are all examples of parameters that are affected by magnesium concentration. (See, Innis et al.). Amplification reactions should contain about a 0.5 to 2.5 mM magnesium concentration excess over the concentration of dNTPs. The presence of magnesium chelators in the reaction can affect the optimal magnesium concentration. A series of amplification reactions can be carried out over a range of magnesium concentrations to determine the optimal magnesium concentration. The optimal magnesium concentration can vary depending on the nature of the target nucleic acid(s) and the primers being used, among other parameters.

5. Deoxynucleotide Triphosphate Concentration

Deoxynucleotide triphosphates (dNTPs) are added to the reaction to a final concentration of about 20 μM to about 300 μM. Typically, each of the four dNTPs (G, A, C, T) are present at equivalent concentrations. (See, Innis et al.).

6. Nucleic Acid Polymerase

A variety of DNA dependent polymerases are commercially available that will function using the methods and compositions of the present invention. For example, Taq DNA Polymerase may be used to amplify target DNA sequences. The PCR assay may be carried out using as an enzyme component a source of thermostable DNA polymerase suitably comprising Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g., Thermus and Pyrococcus species. Concentration ranges of the polymerase may range from 1-5 units per reaction mixture. The reaction mixture is typically between 15 and 100 μl.

In some embodiments, a “hot start” polymerase can be used to prevent extension of mispriming events as the temperature of a reaction initially increases. Hot start polymerases can have, for example, heat labile adducts requiring a heat activation step (typically 95° C. for approximately 10-15 minutes) or can have an antibody associated with the polymerase to prevent activation.

7. Other Agents

Additional agents are sometime added to the reaction to achieve the desired results. For example, DMSO can be added to the reaction, but is reported to inhibit the activity of Taq DNA Polymerase. Nevertheless, DMSO has been recommended for the amplification of multiple target sequences in the same reaction. (See, Innis et al. supra). Stabilizing agents such as gelatin, bovine serum albumin, and non-ionic detergents (e.g. Tween-20) are commonly added to amplification reactions. (See, Innis et al. supra).

D. Amplification

Amplification of an RNA or DNA template using reactions is well known (see, U.S. Pat. Nos. 4,683,195 and 4,683,202; PCR PROTOCOLS: A GUIDE TO METHODS AND APPLICATIONS (Innis et al., eds, 1990)). Methods such as polymerase chain reaction (PCR) and ligase chain reaction (LCR) can be used to amplify nucleic acid sequences of target DNA sequences directly from samples such as a bacterial isolate or soil sample. The reaction is preferably carried out in a thermal cycler to facilitate incubation times at desired temperatures. As discussed above, degenerate oligonucleotides can be designed to amplify target DNA sequence homologs using the known sequences that encode the target DNA sequence. Restriction endonuclease sites can be incorporated into the primers.

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 15 seconds-2 minutes, an annealing phase lasting 10 seconds.-2 minutes, and an extension phase of about 72° C. for 5 seconds-2 minutes.

In some instances, the amplification reaction is a nested PCR assay as described in, e.g., Bender, K. S. et al., Appl. Environ. Microbiol., 70: 5651-5658 (2004). Two amplification steps are carried out. The first amplification uses an “outer” pair of primers designed to amplify a highly conserved region of the target sequence. The second amplification uses an “inner” (i.e., “nested”) pair of primers designed to amplify a portion of the target sequence that is contained within the first amplification product.

Isothermic amplification reactions are also known and can be used according to the methods of the invention. Examples of isothermic amplification reactions include strand displacement amplification (SDA) (Walker, et al. Nucleic Acids Res. 20(7):1691 (1992); Walker PCR Methods Appl 3(1):1 (1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), and branched DNA signal amplification (bDNA) (see, e.g., Iqbal et al., Mol. Cell Probes 13(4):315 (1999)). In a preferred embodiment, rolling circle amplification (RCA) (Lisby, Mol. Biotechnol. 12(1):75 (1999)); Hatch et al., Genet. Anal. 15(2):35 (1999)) is used. Other amplification methods known to those of skill in the art include CPR (Cycling Probe Reaction), SSR (Self-Sustained Sequence Replication), SDA (Strand Displacement Amplification), QBR (Q-Beta Replicase), Re-AMP (formerly RAMP), RCR (Repair Chain Reaction), TAS (Transcription Based Amplification System), and HCS (hybrid capture system). Any amplification method known to those of skill in the art may be used with the methods of the present invention provided two primers are present at either end of the target sequence.

F. Detection of Amplified Products

Any method known in the art can be used to detect the amplified products, including, for example solid phase assays, anion exchange high-performance liquid chromatography, and fluorescence labeling of amplified nucleic acids (see MOLECULAR CLONING: A LABORATORY MANUAL (Sambrook et al. eds. 3d ed. 2001); Reischl and Kochanowski, Mol. Biotechnol. 3(1): 55-71 (1995)). Gel electrophoresis of the amplified product followed by standard analyses known in the art can also be used to detect and quantify the amplified product. Suitable gel electrophoresis-based techniques include, for example, gel electrophoresis followed by quantification of the amplified product on a fluorescent automated DNA sequencer (see, e.g., Porcher et al., Biotechniques 13(1): 106-14 (1992)); fluorometry (see, e.g., Innis et al., supra), computer analysis of images of gels stained in intercalating dyes (see, e.g., Schneeberger et al., PCR Methods Appl. 4(4): 234-8 (1995)); and measurement of radioactivity incorporated during amplification (see, e.g., Innis et al., supra).

In an embodiment, dual labeled probes are used to detect the amplified DNA. Dual labeled probes include e.g., probes labeled with both a reporter and a quencher dye, which fluoresce only when bound to their target sequences and pairs of probes, each of which is labeled with a reporter and quencher day. In some embodiments, the dual labeled probes use fluorescence resonance energy transfer (FRET) technology in which probes labeled with either a donor or acceptor label bind within the amplified fragment adjacent to each other, fluorescing only when both probes are bound to their target sequences. Suitable reporters and quenchers include, for example, black hole quencher dyes (BHQ), TAMRA, FAM, CY3, CY5, Fluorescein, HEX, JOE, LightCycler Red, Oregon Green, Rhodamine, Rhodamine Green, Rhodamine Red, ROX, TAMRA, TET, Texas Red, and Molecular Beacons.

In another embodiment, FRET probes and primers can be used to detect the pcrA gene. One of skill in the art will appreciate that the primers and probes can conveniently be designed for use with the Lightcycler system (Roche Molecular Biochemicals). For example, a single set of primers (e.g., pcrA320F and pcrA598R) and probes specific for particular pcrA genes, internal to the sites of the PCR primers, can conveniently be designed so that the DNA from multiple forms of the pcrA gene (e.g., those from different bacterial species) would amplify, and the probes would bind to the different amplicons depending on the bacterial origin of the pcrA gene. Furthermore, probes specific non-pcrA reductases, such as members of the DMSO reductase family, can be used as controls to monitor the amplification of non-pcrA sequences.

In a further embodiment, RealTime PCR is used to detect target sequences. For example, in such an embodiment, Real-time PCR using SYBR® Green I can be used to amplify and detect the target nucleic acids (see, e.g., Ponchel et al., BMC Biotechnol. 3:18 (2003)). SYBR® Green I only fluoresces when bound to double-stranded DNA (dsDNA). Thus, the intensity of the fluorescence signal depends on the amount of dsDNA that is present in the amplified product. Specificity of the detection can conveniently be confirmed using melting curve analysis.

A preferred method of PCR amplification and detection is quantitative real time PCR (qPCR or qRT-PCR). This method allows the simultaneous quantitation and amplification of a specific subsequence of a DNA molecule. Thus, this method allows the user to not only determine whether or not a DNA is present in the sample, but also the number of copies of the sample, in real time. Thus qPCR follows the general methodology of standard PCR but the DNA is quantitated after each round of amplification. The two most common methods of quantitation utilize either fluorescent dyes that intercalate with double-stranded DNA or by use of modified DNA oligonucleotide probes that fluoresce when hybridized with a complimentary DNA (see, e.g., Innis et al.; Heid et al., Genome Methods, 6: 986-994 (1996)). Instrumentation and reagents for qPCR are commercially available, such as the 7300 real-time PCR system (Applied Biosystems, Foster City, Calif.) and its associated reagents.

The amplification and detection steps can be carried out sequentially, or simultaneously.

G. Sequence Specific Hybridization

In addition to the nucleic acid amplification methods described above, the detection and quantitation of PRB in a sample can be determined by measuring the amount of a sequence-specific oligonucleotide that hybridizes to the unamplified or amplified pcrA gene in the sample. Typically, the oligonucleotide is labeled with a detectable label, for example a fluorescent label, and applied to a sample that is to be tested for the presence of PRB. After stringent hybridization and washing conditions, fluorescence intensity bound to the sample is measured.

Suitable assay formats for detecting hybrids formed between oligonucleotide probes and target nucleic acid sequences in a sample are known in the art and include the immobilized target (dot-blot) format and immobilized probe (reverse dot-blot or line-blot) assay formats or slot blot formats. Dot blot and reverse dot blot assay formats are described in U.S. Pat. Nos. 5,310,893; 5,451,512; 5,468,613; and 5,604,099; each incorporated herein by reference.

In a dot-blot or slot blot format, a target DNA is immobilized on a solid support, such as a nylon membrane. The membrane-target complex is incubated with labeled oligonucleotide probe under suitable hybridization conditions, unhybridized probe is removed by washing under suitably stringent conditions, and the membrane is monitored for the presence of bound probe.

In the reverse dot-blot (or line-blot) format, the oligonucleotide probe is immobilized on a solid support, such as a nylon membrane or a microtiter plate. The target DNA is labeled, typically during amplification by the incorporation of labeled primers. One or both of the primers can be labeled. The membrane-probe complex is incubated with the labeled amplified target DNA under suitable hybridization conditions, unhybridized target DNA is removed by washing under suitably stringent conditions, and the membrane is monitored for the presence of bound target DNA.

### V. Compositions and Kits of the Invention

The compositions of the invention comprise primers capable of hybridizing to a subsequence of the pcrA gene. Typically, the compositions of the invention provide at least a pair of primers which may be used to amplify a subsequence of the pcrA gene from a sample.

The present invention also provides kits for hybridization detection and/or amplification of the pcrA gene from a sample. Such kits typically comprise two or more components necessary for amplifying the pcrA gene. Components may be compounds, reagents, containers and/or equipment. For example, one container within a kit may contain a set of primers, e.g., pcrA320F and pcrA598R; and another container within a kit may contain a optionally a second set of primers, e.g., for nested PCR or for detection of a sequence internal to the first set of primers. In addition, kits may contain a DNA polymerase suitable for amplification of a subsequence of the pcrA gene using the primers of the kit. In addition, the kits comprise instructions for use, i.e., instructions for using the primers in amplification and/or detection reactions as described herein.

### Example 1

### Primer Design

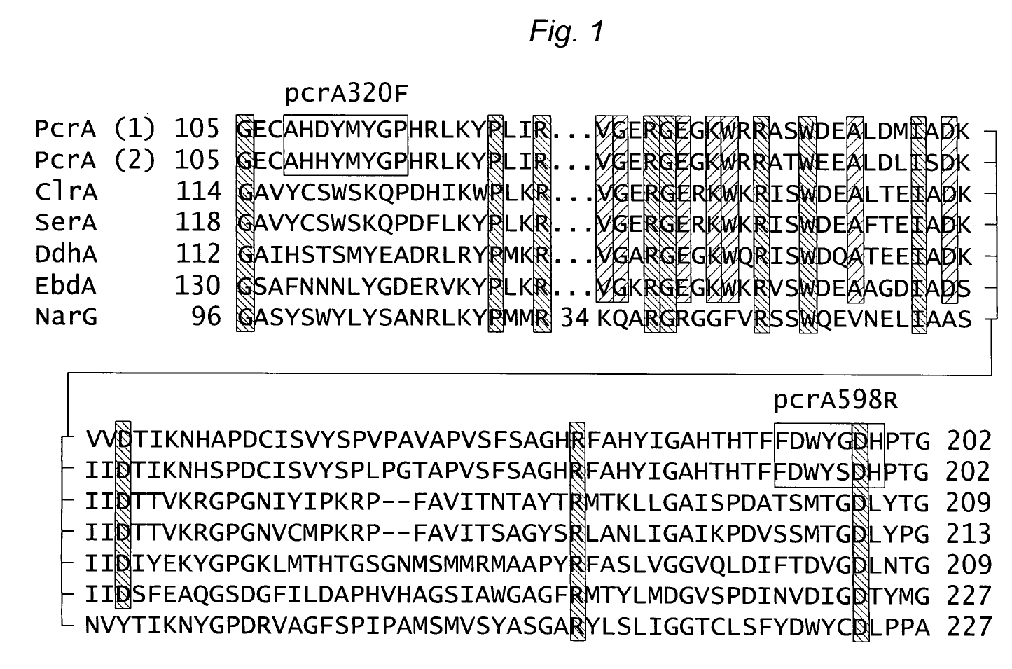

To identify conserved regions, deduced PcrA protein sequences from Dechloromonas agitata CKB (Genbank accession AAO49008) and D. aromatica RCB (AAZ47315, http://genome.jgi-psf.org/finished_microbes/decar/decar.home.html) were aligned using Clustal W (Thompson, J. et al., Nucl. Acids. Res., 22:4673-4680 (1994)) (FIG. 1). Several other molybdoenzyme sequences from the dimethyl sulfoxide (DMSO) reductase family were included in order to identify unique PcrA sequence regions (FIG. 1). This enzyme group includes those having important roles in anaerobic respiration, in particular, respiratory reduction of oxyanions, such as nitrate, selenate, arsenate, and chlorate, in addition to perchlorate (McEwan, A. G. et al., Geomicrobiol. J, 19:3-21 (2002)).

A primer pair, pcrA320F (5′-GCGCCCACCACTACATGTAYGGNCC-3′) and pcrA598R (5′-GGTGGTCGCCGTACCARTCRAA-3′), was selected using CODEHOP (Thorell, H. D. et al., Appl. Environ. Microbiol., 69:5585-5592 (2003)) along with inspection of the sequences and the degrees of genetic code degeneracy. The primer sequences correspond to nucleotide positions 320-344 and 577-598 of the D. agitata CKB pcrA gene.

### Example 2

### Detection of pcrA Genes in Perchlorate-Reducing Cultures

Detection of PRB by qPCR using the designed pcrA primers (pcrA-qPCR) was confirmed with DNA from pure cultures of five PRB strains from four genera, Dechloromonas, Azospira, Azospirillum, and Dechlorospirillum, representing most of the previously-identified PRB (Bruce, R. A. et al., Environ. Microbiol., 1:319-329 (1999); Coates, J. D. et al., Appl Environ. Microbiol., 65:5234-5241 (1999)). The assay was also tested with DNA extracted from Yolo silt loam soil enriched with 0.25 mM perchlorate and either acetate (YA) or hydrogen (YH) provided as electron donors (Nozawa-Inoue, M. et al., Appl. Environ. Microbiol., 71:3928-3934 (2005)). Two non-PRB were also tested, including a chlorate-reducing Pseudomonas sp. PK (presumably containing the clrA gene encoding the molybdoenzyme chlorate reductase) (Bender, K. S. et al., J. Bacteriol., 187:5090-5096 (2005); Coates, J. D. et al., Appl. Environ. Microbiol., 65:5234-5241 (1999)) and a nitrate-reducing Escherichia coli K-12 (which encodes several molybdoenzymes).

Pure culture and soil enrichment DNA was extracted using UltraClean Microbial DNA kit (MoBio laboratories, Calsbad, Calif.) and FastDNA Spin kit for soil (MP Biomedicals, Solon, Ohio), respectively. Five ng of each DNA sample was added to a qPCR mixture (15 μl as a final reaction volume) containing 1×SYBR Ex Taq premix (TaKaRa Bio USA, Madison, Wis.) and primers (0.2 μM each). PCR was performed by a 7300 real-time PCR system (Applied Biosystems, Foster City, Calif.) with a thermal cycling of 95° C. for 1 min followed by 35 cycles of 95° C. for 5 sec and 60° C. for 31 sec. The absence of non-specific PCR products was confirmed both by dissociation curve analysis and by 1.5% agarose gel electrophoresis.

Although the primers were designed using the only two available pcrA sequences, both from genus Dechloromonas, substantial amplifications were also observed from two of the other three PRB, Azospirillum sp. TTI and Dechlorospirillum sp. WD (Table 1). Amplification was not observed with DNA from Azospira suillum PS, nor from the negative controls.

Amplification was also observed in DNA extracted from YA and YH soil enrichment cultures (Table 1), in which 16S rRNA genes identical to Dechlorospirillum sp. and Azospirillum sp. were previously detected (Nozawa-Inoue, M. et al., Appl. Environ. Microbiol., 71:3928-3934 (2005)).

TABLE 1
Detection of pcrA genes with PRB strains, soil enriched
with perchlorate, and non-PRB strains by qPCR

| | Perchlorate | QPCR |
|---|---|---|
| Strains | reduction^{c} | detection^{d} |
| Dechloromonas agitata CKB | + | + |
| Dechlomonas sp. MissR | + | + |
| Azospira suillum PS | + | − |
| Azospirillum sp. TTI | + | + |
| Dechlorospirillum sp. WD | + | + |
| Pseudomonas sp. PK | − | − |
| Escherichia coli K-12 | − | |
| Soil enriched with ClO₄⁻/acetate (YA)^{a} | + | + |
| Soil enriched with ClO₄⁻/H₂ (YH)^{a} | + | + |

^{a}Yolo silt loam soil enriched in a mineral liquid medium containing 2.5 mM ClO₄⁻ with either 10 mM acetate under N₂ headspace (YA) or 10 mM bicarbonate under H₂ headspace (YH) for 4 to 5 months (Nozawa-Inoue, M. et al., Appl. Environ. Microbiol., 71: 3928-3934 (2005))
^{b}Ability to reduce perchlorate to chloride
^{c}Detection above the detection limit (=9 copies/reaction)

### Example 3

### Partial pcrA Gene Sequences

pcrA amplicons from Dechloromonas sp. strains CKB and MissR, Dechlorospirillum sp. WD, Azospirillum sp. TTI, and the soil enrichment cultures, YA and YH, were cloned using TOPO TA cloning kit (Invitrogen, Carlsbad, Calif.). Positive clones were identified following screening with M13 universal primers. For the enrichment cultures, that likely contained multiple strains of PRB, the M13 PCR products of positive clones were subjected to restriction fragment length polymorphisms (RFLP) using the restriction endonuclease Hha I. The digestion patterns were examined by performing gel electrophoresis with 3% low-melting agarose gel (Fisher Scientific, Fair Lawn, N.J.) in 1×TBE buffer at 6V/cm and 4° C. Plasmids were extracted from the pcrA clones with distinct RFLPs and from those of PRB pure cultures using Plasmid Mini kit (Qiagen, Valencia, Calif.). Inserts were sequenced at the UC Davis DNA sequencing facility (Davis, Calif.). The amino acid sequences of the clones are provided in FIG. 2.

The alignment provided in FIG. 2 can be used to design additional primers that can be used to amplify additional subsequences of the pcrA gene, while avoiding the amplification of subsequences from non-perchlorate reductase genes.

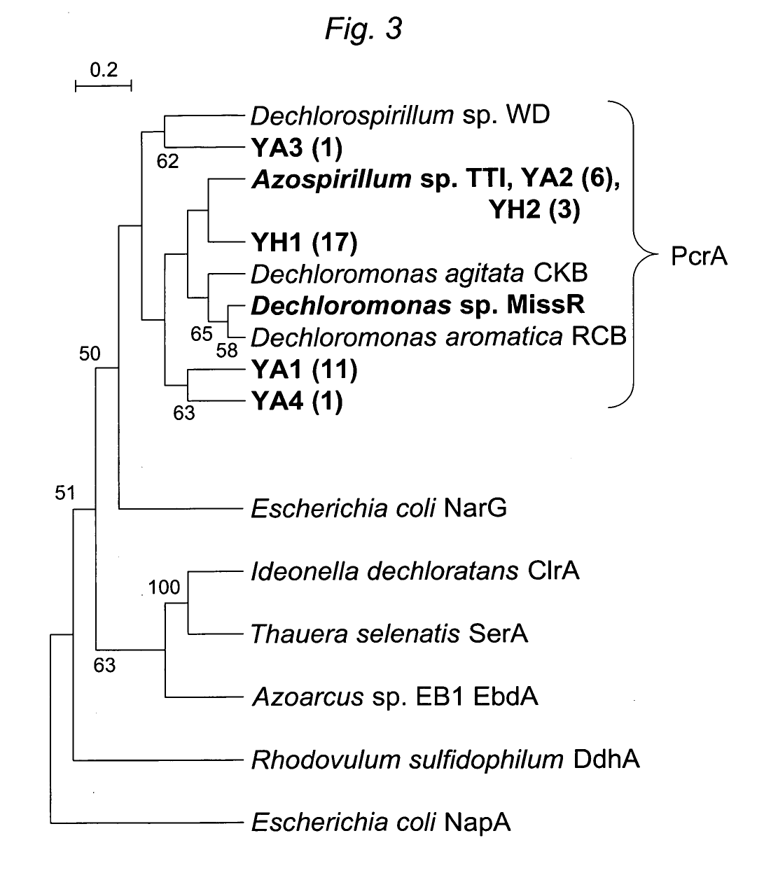

A phylogenetic tree of the deduced PcrA protein sequences (92 amino acid), and the corresponding sequences of the enzymes in the DMSO reductase family, was constructed using the neighbor-joining method (Saitou, N., and M. Nei., Mol. Biol. Evol., 4:406-425 (1987)) (FIG. 3). The PcrA sequences of PRB isolates and soil enrichments were closely related to each other but distinct from other molybdoenzymes in the DMSO reductase family. Within the PcrA cluster, the three Dechloromonas spp. PcrA sequences formed a tight group, indicating that they are closely related.

The enrichment culture PcrA sequences YA3 and YH2 were closely related to those from the Dechlorospirillum and Azospirillum pure cultures (FIG. 2), respectively, as anticipated from the previous identification of the corresponding 16S rRNA gene sequences in these samples (Nozawa-Inoue, M. et al., Appl. Environ. Microbiol., 71:3928-3934 (2005)). In contrast, the YA2 PcrA sequence was identical to that from the Azospirillum sp. pure culture (FIG. 2), although we did not detect Azospirillum sp. 16S rRNA gene sequences in the YA enrichment (Nozawa-Inoue, M. et al., Appl. Environ. Microbiol., 71:3928-3934 (2005)). A correlation of sequence phylogenies between NarG and 16S rRNA gene was previously found using pure culture sequences (Philippot, L., Biochim. Biophys. Acta, 1577:355-376 (2002)), although a study with isolates from a fresh sediment sample showed the correlation was not entirely consistent (Gregory, L. G. et al., Microbiology-(UK), 149:229-237 (2003)).

A total of four and two different PcrA sequences were obtained from the YA and YH, respectively, suggesting relatively higher diversity associated with exposure to acetate than hydrogen (FIG. 2). Only one pcrA clone sequence was identical between the YA and YH enrichments, suggesting different electron donors may enrich different PRB.

### Example 5

### Plasmid Standard Curve for Quantification

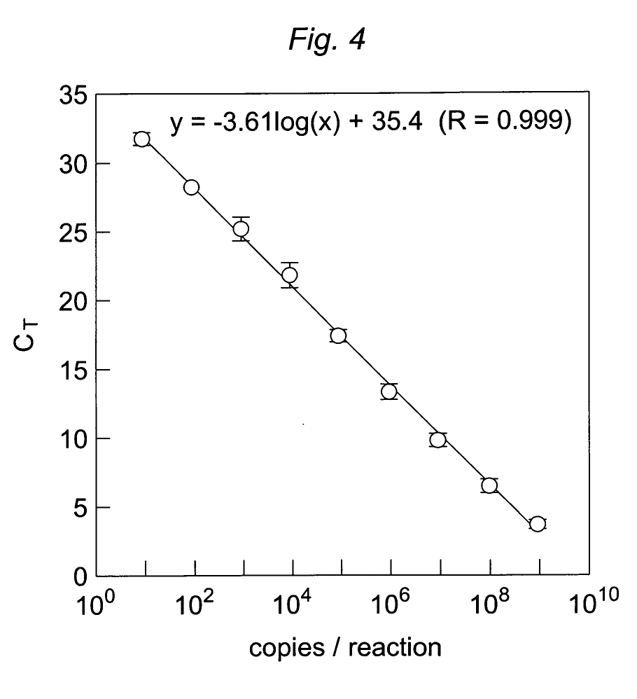

A standard curve was constructed relating gene copy numbers to qPCR threshold cycle (C_{T}) using a plasmid with the cloned D. agitata CKB (ATCC700666) pcrA gene. The copy number of the plasmid was determined by measuring absorbance at 260 nm. Five microliters of 10-fold serial dilutions of the plasmid solution was added to a qPCR mixture, and qPCR was performed as described above. The curve relating gene copy numbers and qPCR threshold cycles was strongly linear (R²=0.99) over 9 orders of magnitude (FIG. 4). The detection limit was approximately 9 copies/reaction.

### Example 6

### Quantification of pcrA Genes in Soil Samples

The pcrA genes were amplified in samples of a previously unexposed Yolo silt loam soil and in an undescribed soil collected from a perchlorate-contaminated site in California (Soil B). In addition, two sets of anaerobic unsaturated microcosms using these two soils were exposed to perchlorate and amended with either acetate or hydrogen as an electron donor. Approximately 1.0 and 0.2 μmol/g-dry soil of perchlorate was reduced in the Yolo soil microcosms and the Soil B microcosms, respectively, in the presence of either acetate and nitrogen gas (Yolo soil only) or bicarbonate and hydrogen gas (both Yolo soil and Soil B) (Nozawa-Inoue, M. et al., Appl. Environ. Microbiol., 71:3928-3934 (2005), unpublished data). Five microliters of soil DNA, diluted 100 times, was analyzed by qPCR. The dilution served to reduce the inhibitory effect from the sample matrices in order to maximize qPCR amplification. The pcrA copy numbers were calculated based on the standard curve described above. Although pcrA genes were not detected in any of the soils before treatment, 10⁴-10⁵ copies of pcrA genes per gram dry soil were successfully detected in samples after perchlorate reduction (Table 2), presumably due to the growth of PRB.

TABLE 2
pcrA copy numbers in soil microcosm samples.

| Soil | Treatment | pcrA copies/g dry soil |
|---|---|---|
| Yolo silt loam | Untreated | <DL^{a} |
| | ClO₄⁻/acetate | 3.4 ± 2.1 × 10⁴ |
| | ClO₄⁻/H₂ | 9.6 ± 6.0 × 10⁴ |
| Soil B | Untreated | <DL^{a} |
| | ClO₄⁻/H₂ | 4.5 ± 2.7 × 10⁵ |

^{a}Under the detection limit

Thus, the qPCR assay of the present invention described herein allows the quantitation of the abundance of pcrA genes, reflecting the PRB population, in environmental samples. The sequence information collected using the pcrA gene primers also suggests the enrichment of pcrA sequences specific for the environmental conditions. The assay may be used to estimate cell densities of PRB involved in perchlorate reduction in microbial communities and may be used for optimizing biological treatment of perchlorate, including bioreactors and in-situ bioremediation.

What is claimed is:

1. A method of detecting perchlorate-reducing bacteria in a sample, the method comprising contacting the sample with an oligonucleotide that specifically hybridizes to a subsequence of the pcrA gene, whereby specific hybridization to the subsequence indicates the presence of perchlorate-reducing bacteria in the sample.

2. The method of claim 1, wherein the oligonucleotide comprises pcrA320F or pcrA598R.

3. The method of claim 1, wherein the oligonucleotide comprises pcrA320F and pcrA598R.

4. The method of claim 1, wherein the method of detecting is carried out using PCR.

5. The method of claim 4, wherein the PCR is real-time quantitative PCR.

6. The method of claim 1, wherein the oligonucleotide comprises a detectable label.

7. The method of claim 6, wherein the detectable label is a fluorescent label.

8. The method of claim 1, wherein the sample comprises a soil sample.

9. A composition for detecting perchlorate-reducing bacteria in a sample, the composition comprising an oligonucleotide that specifically hybridizes to a subsequence of the pcrA gene.

10. The composition of claim 9, wherein the oligonucleotide comprises pcrA320F or pcrA598R.

11. The composition of claim 9, wherein the oligonucleotide comprises pcrA320F and pcrA598R.

12. The composition of claim 9, wherein the oligonucleotide comprises a detectable label.

13. The composition of claim 12, wherein the detectable label is a fluorescent label.

14. A kit for detecting perchlorate-reducing bacteria in a sample, the kit comprising an oligonucleotide that specifically hybridizes to a subsequence of the pcrA gene.

15. The kit of claim 14, wherein the oligonucleotide comprises pcrA320F or pcrA598R.

16. The kit of claim 14, wherein the oligonucleotide comprises pcrA320F and pcrA598R.

17. The kit of claim 14, wherein the oligonucleotide comprises a detectable label.

18. The kit of claim 17, wherein the detectable label is a fluorescent label.

19. The kit of claim 14, further comprising a DNA polymerase.

20. The kit of claim 19, wherein the DNA polymerase is Taq polymerase.
